(19) United States
(12) Patent Application Publication
(10) Pub. No.: US 2014/0259209 A1
(43) Pub. Date: Sep. 11, 2014

(54) ROOT SOAKING METHOD TO PRODUCE DOUBLE HAPLOID INBRED LINES IN CORN

(71) Applicant: Dow AgroSciences LLC, Indianapolis, IN (US)

(72) Inventors: Roberto V. Carvalho, Ribeirao Preto-Sp (BR); Edimilson Linares, Ribeirao Preto-Sp (BR); Silvio Piovan, Ribeirao Preto (BR); Jose D. Leon, Zionsville, IN (US)

(73) Assignee: Dow AgroSciences LLC, Indianapolis, IN (US)

(21) Appl. No.: 14/197,256

(22) Filed: Mar. 5, 2014

Related U.S. Application Data
(60) Provisional application No. 61/774,615, filed on Mar. 8, 2013.

(51) Int. Cl. C12N 15/01

(52) U.S. Cl. CPC C12N 15/01
USPC 800/270; 800/276

(57) ABSTRACT

The subject invention pertains to a method of generating double haploid (DH) corn inbred lines from haploid corn plants. The roots of haploid corn plants are contacted with a solution/composition comprising about 0.0015M colchicine and about 0.11M dimethyl sulfoxide and the treated haploid corn plants are grown. DH corn plants are selected from the treated haploid corn plants and the selected DH corn plants are used to produce DH corn inbred lines. The present invention also provides a method for increasing the DH success rate with corn plants.

This application claims a priority based on provisional application 61/774,615 which was filed in the U.S. Patent and Trademark Office on Mar. 8, 2013, the entire disclosure of which is hereby incorporated by reference.

### BACKGROUND OF THE INVENTION

Corn breeders use inbred line development to generate genetically homozygous lines to produce superior hybrids. The conventional method to produce homozygous lines is self-pollination which typically requires about eight generations. This method is time consuming and may take four or more years. Doubled haploid (DH) technology reduces the homozygous inbred line development cycle-time by about two years with a concomitant acceleration in hybrid creation and commercial launching.

The Root Soaking (RS) method disclosed in this application provides a novel approach in DH technology. Under the RS method, after treatment according to the current invention the haploid corn plants are planted in fields, eliminating the intermediate tray planting step. This reduces labor and time. Also, under the Seedling Soaking (SS) method, young seedlings are treated with colchicine. Since colchicine is toxic to plants, treating young seedlings with colchicine kills a high percentage of treated seedlings. Under the disclosed RS method, corn plants are treated with colchicine at a later stage of development when they are stronger. Therefore, colchicine treatment does not kill many corn plants and the percentage of haploid plants surviving the colchicine treatment is higher.

### BRIEF SUMMARY OF THE INVENTION

The present invention relates to a method of generating DH inbred lines from haploid corn plants. The roots of haploid corn plants are contacted with a solution/composition comprising about 0.0015M colchicine and about 0.11M dimethyl sulfoxide (DMSO).

In yet another aspect of the invention, the haploid corn plants treated according to the current invention are haploid corn seedlings. The haploid corn seedlings may be obtained by germinating haploid corn seeds. An aspect of the invention also involves growing the treated haploid corn seedlings into mature corn plants and selecting DH corn plants from the mature corn plants.

In another aspect of the invention, treating the haploid corn plants according to the current invention causes chromosome doubling in the treated haploid corn plants which produces DH corn plants. In certain embodiments, chromosome doubling occurs in about 10% to about 25% of the treated haploid corn plants. The treated haploid corn plants are grown further and DH corn plants identified. Once identified, the DH corn plants are selected and grown further. DH corn plants may be treated with a composition comprising zinc and boron, preferably prior to flowering, and self-pollinated. The corn seeds produced by the self-pollinated DH corn plants may also be collected. In a further aspect of the invention, the corn seeds obtained from the DH corn plants are used to generate DH inbred corn lines.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIG. 1. Planted haploid corn seeds on the left and growing haploid corn seedlings on the right.

FIG. 2. Comparison between SS and RS method. Treating young corn seedlings with colchicine under the SS method on the left and treating older and more mature haploid corn seedlings with the colchicine and DMSO solution/composition on the right.

FIG. 3. Comparison between SS and RS methods steps showing the operational advantage of RS method, reducing one labor step.

FIG. 4. Haploid corn seedlings after treatment with the colchicine and DMSO solution of the current invention on the left and haploid corn plants planted in the field under ALUMINET cover shade on the right.

### DETAILED DISCLOSURE OF THE INVENTION

Various aspects and embodiments of this invention are directed to methods for obtaining doubled haploid corn plants or corn plant components. The method utilizes a root soaking technique wherein the roots of corn plants or corn seedlings are contacted with a solution/composition comprising about 0.0015M colchicine and about 0.11M DMSO.

A doubled haploid (“DH”) or a doubled haploid corn plant refers to a plant developed by the doubling of a haploid set of chromosomes. A doubled haploid corn plant is considered a homozygous corn plant. A corn plant is considered to be doubled haploid even if the entire vegetative part of the corn plant does not contain cells with a doubled set of chromosomes; however, a doubled haploid corn plant must be fertile.

The term “about” is used in this patent application to describe some quantitative aspects of the invention, for example, concentration of chemicals or time of contact. It should be understood that absolute accuracy is not required with respect to those aspects for the invention to operate. When the term “about” is used to describe a quantitative aspect of the invention the relevant aspect may be varied by ±10%. As would be apparent to those skilled in the art, ±10% permits the quantitative element (e.g., the concentration of colchicine or DMSO) to be varied by ±1%, ±2% , ±3% , ±4%, ±5%, ±6% , ±7% ±8%, ±9% or ±10%).

Thus, one aspect of the invention provides a method for generating a DH corn plant comprising contacting the roots of a haploid corn plant or a haploid corn seedling with a solution/composition comprising about 0.0015M colchicine and about 0.11M DMSO.

Haploid corn plants or haploid corn seedlings for use in the disclosed methods may be obtained from haploid corn plants or germinated from corn seeds grown in soil (or containers of soil). By way of example, soil can include a composition comprising an organic substrate and superfine vermiculite in equal proportion. Haploid corn plants or corn seedlings are removed from a container or soil after sufficient growth is achieved and the roots of the haploid corn plants or corn seedlings are cleaned, for example, by washing with water. Haploid corn plants or corn seedlings are then treated with a composition/solution comprising about 0.0015M colchicine and about 0.11M DMSO. The roots of haploid corn plants or corn seedlings are substantially submerged in the colchicine and DMSO composition/solution for a period of up to 24 hours. For example, the roots of the haploid corn plants or haploid corn seedlings can be submerged in the colchicine and DMSO composition/solution for a period of about 10 to about 20 hours or for about 15 hours. After the haploid corn plants or haploid corn seedlings are soaked for a desired amount of time the treated haploid corn plants or haploid corn seedlings are planted to facilitate further growth. In certain embodiments, the treated haploid corn plants or treated corn haploid seedlings are washed with water or another solution to remove residual colchicine and DMSO from the roots of the treated haploid corn plants or haploid corn seedlings.

In certain embodiments of the invention, the treated haploid corn plants or haploid corn seedlings are then planted in a field under a shade cover, for example, an ALUMINET shade cloth, for further growth. ALUMINET is a knitted/woven fabric made of mono-oriented high-density polyethylene (HDPE) fibers which have been metalized with aluminum. ALUMINET shade cloth may also be coated with an anti-oxidation layer meeting the ASTN-D-3887 standard. Environmental conditions, for example, light, temperature, availability of water and nutrients, and humidity may be controlled for optimum growth and development of the treated haploid plants or haploid seedlings. However, other equivalent forms of shade cover (e.g., other types of shade cloth used in the horticulture industry) can also be used.

Chromosome doubling occurs in haploid corn plants or haploid corn seedlings after treatment with the colchicine and DMSO composition/solution. If chromosome doubling occurs in a treated corn haploid plant or haploid corn seedling, a DH corn plant may be produced. As discussed in the Examples, the disclosed method improves the success index (SI) of obtaining DH corn plants. In the context of this application, the success index (SI) is: the number of double haploid (DH) lines obtained out of 100 PHK (Putative Haploid Kernels) expressed as a percentage. For example, if one obtains 1130 DH corn lines from 10,000 PHK, the SI is 11.3% (SI=(1130 DH corn lines/10,000 PHK)*100). The success index for the disclosed method ranges between about 10% to about 25%.

In another aspect of the invention, DH corn plants are identified from the treated corn plants grown in soil. The DH corn plants may also be selected for further treatment or use in corn breeding programs. During growth, DH corn plants may, optionally, be treated with a composition comprising zinc and boron, optionally, prior to flowering and the zinc/boron composition may be applied to the plants as a foliar fertilizer. In certain embodiments, DH corn plants may be treated with a composition comprising zinc and boron as a liquid formulation using a mechanical sprayer. In various embodiments, the composition applied to the DH corn plants comprises about 7% zinc and about 10% boron.

A further aspect of the invention involves self-pollination of the DH corn plants. Self-pollination of the DH corn plants may produce DH corn seeds. Thus, a further aspect of the invention involves collecting the DH corn seeds from the DH corn plants.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLE 1

### Producing DH Corn Plants and Inbred Lines

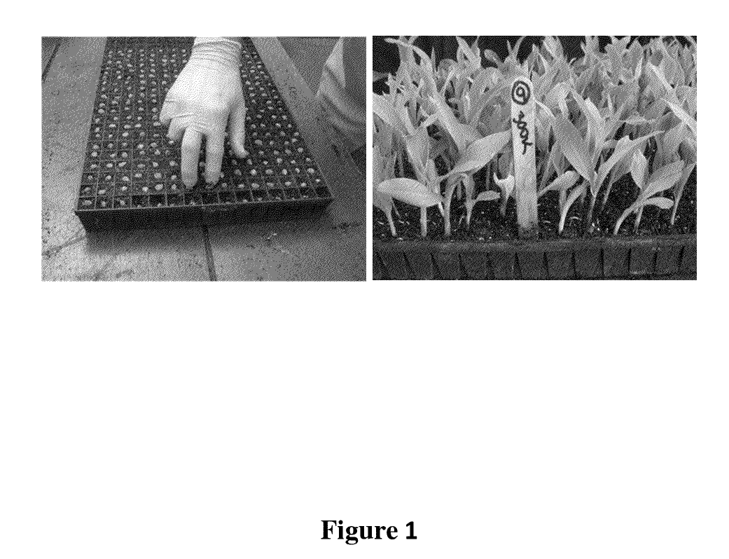

Putative Haploid Kernels (PHK) were obtained through haploid induction of breeding corn plants. PHK are treated with fungicides and insecticides to obtain a uniform and vigorous initial seedling and root development. PHKs are placed in trays containing a composition comprising organic substrate and superfine vermiculite in equal proportion (FIG. 1). The trays are kept in greenhouse in controlled environment providing optimal light, temperature, availability of water and nutrients, and humidity.

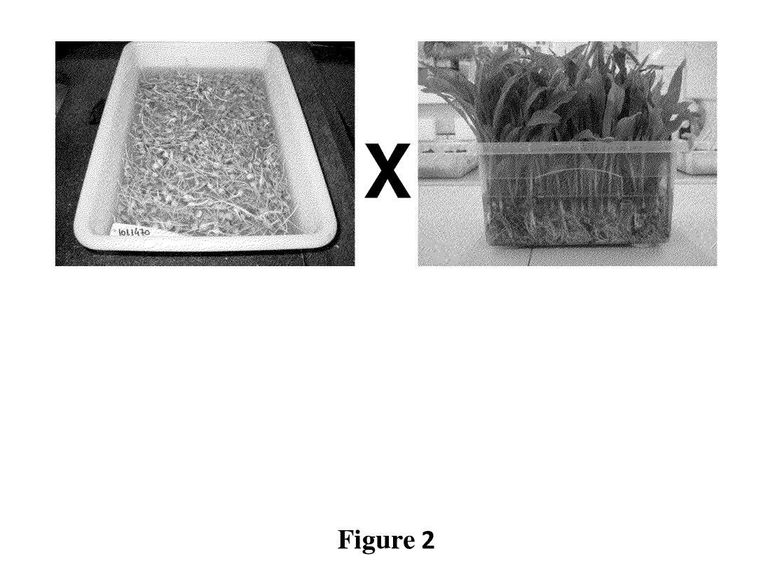

About eight days after planting, haploid seedlings are carefully removed from the trays. Their roots are submerged and stirred in water to ensure proper cleaning. The haploid seedlings are placed in trays. A solution/composition having about 0.0015M colchicine and about 0.11M DMSO is added to the trays, ensuring that the roots of the haploid seedlings are substantially submersed in the solution (FIG. 2).

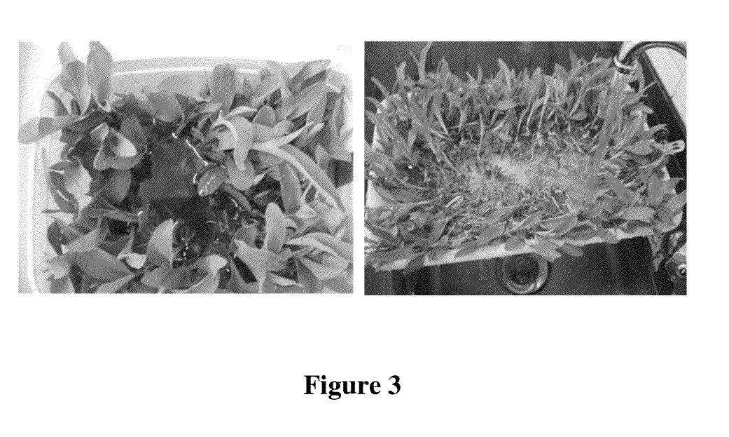

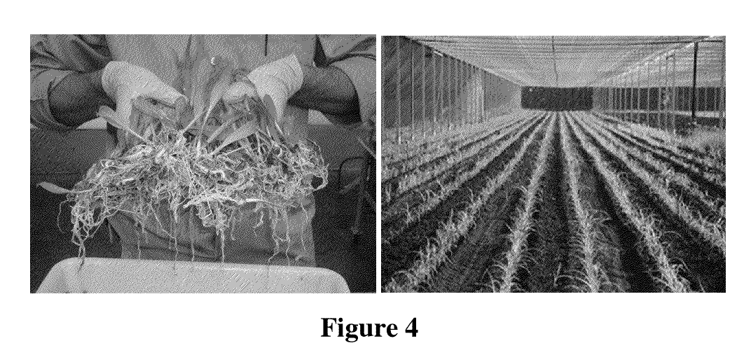

After about 15 hours of soaking, the colchicine and DMSO composition/solution is removed from the trays. The haploid seedlings are washed in running water for about 20 minutes (FIG. 3). The treated haploid seedlings are then planted in fields under an ALUMINET cover shade (FIG. 4) using a two row machine. Two micronutrients, zinc and boron were applied to the planted seedlings about 50 days after planting (as a foliar fertilizer using a mechanical sprayer in a concentration of about 7% Zn and 10% Bo). The plants were self-pollinated and the seeds from the plants collected.

Table 1 shows the comparison between SS method commonly used to generate DH inbred lines and RS method of the current invention. The table provides the data obtained from a large number of starting PHK. The table shows that the RS method provided higher yields compared to the SS method during every step in the process of generating DH inbred lines. Specifically, the RS method produced higher percentage of haploid plants from PHK compared to the SS method (64% vs. 39%; the RS method was less toxic to the seedlings). Further, the RS method produced higher percentage of harvested ears from PHK compared to the SS method (5.8% vs. 13.3%). Finally, the RS method produced higher percentage of selected ears from haploid plants as compared to the SS method (14.8% vs. 20.8%). Conclusively, the RS method provided higher yield of haploid plants from PHK and produced higher numbers of DH inbred lines from PHK.

TABLE 1
Comparison of DH inbred line generation and haploid plant
generation between the SS and the RS method.

| | Number | Number | | | | % |
|---|---|---|---|---|---|---|
| | of | of | Number | | % | Harvested |
| | Putative | Haploid | of | Number | Harvest- | Ears from |
| | Haploid | Plants | Har- | of | ed Ears | Haploid |
| | Kernels | Gener- | vested | Selected | from | plants |
| Treatment | (PHK) | ated | Ears | Ears | PHK | generated |
| SS method | 24731 | 10350 | 1793 | 1790 | 7.3 | 17.3 |
| | 17197 | 6959 | 926 | 886 | 5.4 | 13.3 |
| | 22725 | 7913 | 1013 | 957 | 4.5 | 12.8 |
| Average | 64653 | 25222 | 3732 | 3633 | 5.8 | 14.8 |
| RS method | 5500 | 3240 | 614 | 610 | 11.2 | 19.0 |
| | 24387 | 16261 | 4265 | 4037 | 17.5 | 26.2 |
| | 29354 | 18458 | 3012 | 2958 | 10.3 | 16.3 |
| Average | 59241 | 37959 | 7891 | 7605 | 13.3 | 20.8 |

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A method for generating a doubled haploid corn plant comprising contacting the roots of a haploid corn plant with a solution/composition comprising about 0.0015M colchicine and about 0.11M dimethyl sulfoxide (DMSO) to generate a double haploid corn plant.

2. The method of claim 1, wherein said method comprises contacting the roots of said haploid corn plant with said solution/composition for a period of up to 24 hours.

3. The method of claim 2, wherein the roots of said haploid corn plant are contacted for a period of about 10 to about 20 hours.

4. The method of claim 3, wherein the roots of said haploid corn plant are contacted with said solution/composition for a period of about 15 hours.

5. The method of any one of claims 1-4, wherein said method comprises washing the roots of said haploid corn plant before and/or after the roots of said corn plant are contacted with said solution/composition.

6. The method of claim 1, wherein the method produces a population of haploid corn plants having a success index of about 10% to about 25%.

7. The method of claim 1, wherein the haploid corn plant is a corn seedling.

8. The method of claim 1, further comprising planting said corn plant contacted with said solution/composition.

9. The method of claim 8, said method further comprising selecting double haploid corn plants among said planted corn plants.

10. The method of claim 9, said method further comprising self-pollinating said selected double haploid corn plants.

11. The method of claim 10, said method further comprising generating double haploid seed from said self-pollinated double haploid corn plants.

12. The method of claim 11, said method further comprising collecting double haploid seed from the self-pollinated double haploid corn plants.

13. The method of claim 11 or 12, wherein the double haploid seed comprises a first and a second set of chromosomes that are homozygous.

14. The method of claim 8, said method further comprising self-pollinating said planted corn plants.

15. A method for generating a doubled haploid corn plant comprising:
a) contacting the roots of haploid corn seedlings with a solution/composition comprising about 0.0015M colchicine and about 0.11M dimethyl sulfoxide (DMSO) to generate double haploid corn seedlings;
b) washing said haploid corn seedlings to remove residual solution from the roots of said haploid corn seedlings;
c) planting said washed haploid corn seedlings;
d) growing said planted corn seedlings into mature corn plants; and
e) selecting double haploid corn plants from among the mature corn plants.

16. The method of claim 15, said method further comprising self-pollinating said double haploid corn plants.

17. The method of claim 16, said method comprising collecting seed from said double haploid corn plants.

18. The method of claim 15, wherein said method comprises contacting the roots of said corn seedling with said solution/composition for a period of about 15 hours.

19. The method of claim 15 or 16, said method further comprising applying a fertilizer comprising zinc and boron to said corn plants, optionally, prior to the flowering of said corn plants.

20. The method of any one of claims 15-19, wherein the method produces a population of haploid corn plants and has a success index of about 10% to about 25%.

21. The method of claim 17, wherein said seed comprises a first and a second set of chromosomes that are homozygous.
